(19) United States
(12) Patent Application Publication
(10) Pub. No.: US 2019/0219549 A1
(43) Pub. Date: Jul. 18, 2019

(54) SRM/MRM Assay For The Tubulin Beta-3 Chain (TUBB3) Protein

(71) Applicant: Expression Pathology, Inc., Rockville, MD (US)

(72) Inventors: Todd HEMBROUGH, Gaithersburg, MD (US); Fabiola CECCHI, Washington, DC (US); Sarit SCHWARTZ, Rockville, MD (US)

(21) Appl. No.: 16/330,868

(22) PCT Filed: Sep. 7, 2017
(86) PCT No.: PCT/US2017/050472

§ 371 Date: Mar. 6, 2019

Related U.S. Application Data
(60) Provisional application No. 62/384,202, filed on Sep. 7, 2016.
(60) Provisional application No. 62/402,984, filed on Sep. 30, 2016.

(51) Int. Cl. G01N 30/72; C12Q 1/37; G01N 1/30

(52) U.S. Cl. CPC G01N 30/72; G01N 2001/305; G01N 1/30; C12Q 1/37

(57) ABSTRACT

The current disclosure provides for a specific peptide, and derived ionization characteristics of a peptide from the tubulin beta-3 chain protein (TUBB3) that is particularly advantageous for quantifying the TUBB3 protein directly in biological samples that have been fixed in formalin by the method of Selected Reaction Monitoring (SRM) mass spectrometry. A protein sample is prepared from a biological sample using the Liquid Tissue reagents and protocol and the TUBB3 protein is quantitated by SRM/MRM mass spectrometry analysis of the sample, where the specific peptide is quantitated. Methods of treatment are provided in which the measured level of TUBB3 in a patient tumor sample is compared with a reference level and the patient is treated with a taxane-based treatment regimen when the measured TUBB3 level is lower than the reference level. A suitable reference level is, for example, about 700 amol/μg tissue.

This application claims priority to U.S. Provisional Application Ser. No. 62/384,202, filed Sep. 7, 2016, and to 62/402,984, filed Sep. 30, 2016, both entitled SRM/MRM Assay for the Tubulin beta-3 chain (TUBB3) protein, the contents of each of which are hereby incorporated by reference in their entireties.

### INTRODUCTION

Cancer is treated with a collection of therapeutic agents that function in a variety of ways to kill growing and dividing cells. A common group of chemotherapeutic agents has been used for decades, either individually or in combinations, and have become the traditional and routine cancer treatment in clinical oncology practice. These agents typically act by killing all cells that divide rapidly, one of the main properties of most cancer cells. However, these agents also kill growing normal cells and thus are not considered to be “targeted” approaches to killing cancer cells. In recent years a large group of cancer therapies has been developed that specifically target only cancer cells where the therapeutic agent specifically attacks a protein that is only expressed by the cancer cells and not by normal cells. This approach is considered to be a “targeted” approach to cancer therapy. Most recently, another approach to killing cancer cells in a “targeted” fashion has been to specifically modulate the immune system to enhance the ability of the cancer patient's immune system to kill cancer cells.

Therapeutic agents that target the tubulin beta-3 chain (“TUBB3”) have shown promise in early clinical trials. However, only those patients whose cancer cells express high amounts of the TUBB3 protein are most likely to benefit from treatment with these TUBB3-targeted therapeutic agents. The methods described herein provide a quantitative proteomics-based assay that delivers a relevant measure of activation of the TUBB3 signal pathway as TUBB3 is not normally expressed in normal tissue and/or normal epithelial cells. In particular, the methods provide a mass spectrometry assay that quantifies TUBB3 in formalin fixed tissues from cancer patients and further enables improved treatment decisions for cancer therapy.

A specific peptide derived from subsequences of TUBB3 is provided. The peptide sequence and fragmentation/transition ions for this peptide are useful in a mass spectrometry-based Selected Reaction Monitoring (SRM) assay, which can also be referred to as a Multiple Reaction Monitoring (MRM) assay, and which is referred to herein as SRM/MRM. Use of the peptide for SRM/MRM quantitative analysis of the TUBB3 protein is described.

This SRM/MRM assay can be used to measure relative or absolute quantitative levels of the specific peptides from the TUBB3 protein and provides methods of measuring the amount of the TUBB3 protein in a given protein preparation obtained from a biological sample by mass spectrometry.

More specifically, the SRM/MRM assay can measure the peptide directly in complex protein lysate samples prepared from cells procured from patient tissue samples, such as formalin fixed cancer patient tissue. Methods of preparing protein samples from formalin-fixed tissue are described in U.S. Pat. No. 7,473,532, the contents of which are hereby incorporated by reference in their entirety. The methods described in U.S. Pat. No. 7,473,532 may conveniently be carried out using Liquid Tissue reagents and protocols available from Expression Pathology Inc. (Rockville, Md.).

The most widely available form of tissue from cancer patients is formalin fixed, paraffin embedded tissue. Formaldehyde/formalin fixation of surgically removed tissue is by far the most common method of preserving cancer tissue samples worldwide and is the accepted convention for standard pathology practice. Aqueous solutions of formaldehyde are referred to as formalin. “100%” formalin consists of a saturated solution of formaldehyde (about 40% by volume or 37% by mass) in water, with a small amount of stabilizer (usually methanol) to limit oxidation and degree of polymerization. The most common way in which tissue is preserved is to soak whole tissue for extended periods of time (8 hours to 48 hours) in aqueous formaldehyde, commonly termed 10% neutral buffered formalin, followed by embedding the fixed whole tissue in paraffin wax for long term storage at room temperature. Thus molecular analytical methods to analyze formalin fixed cancer tissue are useful for analysis of cancer patient tissue.

Results from the SRM/MRM assay can be used to correlate accurate and precise quantitative levels of the TUBB3 protein within the specific tissue samples (e.g., cancer tissue sample) of the patient or subject from whom the tissue (biological sample) was collected and preserved. This not only provides diagnostic information about the cancer, but also permits a physician or other medical professional to determine appropriate therapy for the patient. Such an assay that provides diagnostically and therapeutically important information about levels of protein expression in a diseased tissue or other patient sample is termed a companion diagnostic assay. For example, such an assay can be designed to diagnose the stage or degree of a cancer and determine a therapeutic agent to which a patient is most likely to respond.

### SUMMARY

What is provided is a method for measuring the level of the human TUBB3 protein in a human biological sample of formalin-fixed tissue, involving detecting and, advantageously, quantifying the amount of a TUBB3 fragment peptide in a protein digest prepared from the human biological sample using mass spectrometry; and calculating the level of TUBB3 protein in the sample; where the TUBB3 fragment peptide is SEQ ID NO:1, and where the level is a relative level or an absolute level. The protein digest may be fractionated prior to detecting and/or quantifying the amount of the TUBB3 fragment peptide. The protein digest may contain a protease digest, while the tissue may be paraffin-embedded tissue, for example tissue obtained from a tumor.

The TUBB3 fragment peptide may be quantified by comparing the amount of the TUBB3 fragment peptide in one biological sample to the amount of the same TUBB3 fragment peptide in a different and separate biological sample. The TUBB3 fragment peptide may also be quantified by comparison to an added internal standard peptide of known amount, where the TUBB3 fragment peptide in the biological sample is compared to an internal standard peptide having the same amino acid sequence; and where the internal standard peptide is an isotopically labeled peptide.

Detecting and/or quantifying the amount of the TUBB3 fragment peptide in the protein digest can be used to indicate the presence of modified or unmodified TUBB3 protein and an association with cancer in the subject. The results of detecting and/or quantifying the amount of the TUBB3 fragment peptide, or the level of the TUBB3 protein can be correlated to the diagnostic stage/grade/status of the cancer. Correlating the results of the detecting and/or quantifying the amount of the TUBB3 fragment peptide, or the level of the TUBB3 protein to the diagnostic stage/grade/status of the cancer may be combined with detecting and/or quantifying the amount of other proteins or peptides from other proteins in a multiplex format to provide additional information about the diagnostic stage/grade/status of the cancer.

After measuring the level of the human TUBB3 as described above, the patient from whom the biological sample was obtained may be treated with a therapeutically effective amount of a therapeutic agent, where the therapeutic agent and/or amount of the therapeutic agent administered is based upon the amount of the TUBB3 fragment peptide or the level of TUBB3 protein. The therapeutic agents may, for example, bind the TUBB3 protein and/or inhibit its biological activity.

Also provided are methods for treating a patient suffering from cancer involving quantifying the level of a specified TUBB3 fragment peptide, such as the peptide of SEQ ID NO:1, in a protein digest prepared from a tumor sample obtained from the patient and calculating the level of the TUBB3 peptide in the sample by selected reaction monitoring using mass spectrometry, comparing the level of the TUBB3 fragment peptide to a reference level, and either (i) treating the patient with a taxane-based chemotherapy regimen when the level of the TUBB3 fragment peptide is lower than the reference level or (ii) treating the patient with a therapeutic regimen that does not comprise an effective amount of a taxane when the level of the TUBB3 fragment peptide is above the reference level. The reference level may be, for example, 700 amol/μg., +/−250 amol/μg, +/−150 amol/μg, +/−100 amol/μg, +/−50 amol/μg, or +/−25 amol/μg, of biological sample protein analyzed. The protein digest of the biological sample may be prepared by the Liquid Tissue protocol and may comprise a protease digest, such as a trypsin digest.

The patient suffering from cancer may be suffering from gastric cancer and the patient may be treated with FOLFIRI followed by docetaxel and cisplatin (CDDP) when the TUBB3 level is below the cutoff, or may be treated with either FOLFIRI or 5-FU plus folinic acid (leucovorin) when the TUBB3 level is above the cutoff.

The patient suffering from cancer may be suffering triple negative breast cancer, and may be treated with ACT (anthracycline and Cytoxan, followed by taxane) when the TUBB3 level is below the cutoff, or may be treated with CMF (Cytoxan, methotrexate and 5-FU) when the TUBB3 level is above the cutoff.

In any of these methods, the mass spectrometry may be tandem mass spectrometry, ion trap mass spectrometry, triple quadrupole mass spectrometry, MALDI-TOF mass spectrometry, MALDI mass spectrometry, hybrid ion trap/quadrupole mass spectrometry and/or time of flight mass spectrometry. The mode of mass spectrometry used may be Selected Reaction Monitoring (SRM), Multiple Reaction Monitoring (MRM), and/or multiple Selected Reaction Monitoring (mSRM).

In the methods of treatment described above the tumor sample may be a cell, collection of cells, or a solid tissue, and may be formalin fixed solid tissue, including paraffin embedded tissue. In addition, detecting and quantitating the specified TUBB3 fragment peptide can be combined with detecting and quantitating other peptides from other proteins in multiplex so that the treatment decision about which agent used for treatment is based upon specific levels of the specified TUBB3 fragment peptide in combination with other peptides/proteins in the biological sample.

### BRIEF DESCRIPTION OF THE DRAWINGS

FIG. 1 shows that the median overall survival (OS) of gastric cancer patients was significantly shorter for patients with level of TUBB3 above the defined cutoff (>700 amol/ug), than for patients below the cutoff (<700 amol/ug) (1566 vs. 801 days, P=0.0282).

FIG. 2 shows little difference in OS of gastric cancer patients above and below the defined TUBB3 cutoff (>700 amol/ug) for patients treated with FOLFIRU followed by 5-FU plus folinic acid (leucovorin).

FIG. 3 shows significant difference in OS of gastric cancer patients above the defined TUBB3 cutoff (>700 amol/ug) for patients treated with FOLFIRI followed by docetaxel (Taxotere) and cisplatin (CDDP) versus FOLFIRU followed by 5-FU plus folinic acid (leucovorin).

FIG. 4 shows the difference in relapse-free survival (RFS) of triple negative breast cancer patients treated with ACT (anthracycline and Cytoxan, followed by taxane) above and below the cutoff TUBB3 level of 700 amol/ug.

FIG. 5 shows the difference in RFS of triple negative breast cancer patients treated with ACT (anthracycline and Cytoxan, followed by taxane) above and below the cutoff TUBB3 level of 850 amol/ug.

FIG. 6 shows the difference in RFS of triple negative breast cancer patients treated with ACT (anthracycline and Cytoxan, followed by taxane) above and below the cutoff TUBB3 level of 930 amol/ug.

FIG. 7 shows the difference in OS of triple negative breast cancer patients treated with ACT (anthracycline and Cytoxan, followed by taxane) above and below the cutoff TUBB3 level of 930 amol/ug.

### DETAILED DESCRIPTION

The assays described herein measure relative or absolute levels of a specific unmodified peptide from the TUBB3 protein. Relative quantitative levels of the TUBB3 protein can be determined by the SRM/MRM methodology, for example, by comparing SRM/MRM signature peak areas (e.g., signature peak area or integrated fragment ion intensity) of an individual TUBB3 peptide in different samples. Alternatively, it is possible to compare multiple SRM/MRM signature peak areas for multiple TUBB3 signature peptides, where each peptide has its own specific SRM/MRM signature peak, to determine the relative TUBB3 protein content in one biological sample and compare it with the TUBB3 protein content in one or more additional or different biological samples. In this way, the amount of a particular peptide, or peptides, from the TUBB3 protein, and therefore the amount of the TUBB3 protein, is determined relative to the same TUBB3 peptide, or peptides, across two or more biological samples under the same experimental conditions. In addition, relative quantitation can be determined for a given peptide, or peptides, from the TUBB3 protein within a single sample by comparing the signature peak area for that peptide by SRM/MRM methodology to the signature peak area for another and different peptide, or peptides, from a different protein, or proteins, within the same protein preparation from the biological sample. In this way, the amount of a particular peptide from the TUBB3 protein, and therefore the amount of the TUBB3 protein, is determined relative one to another within the same sample.

These approaches generate quantitation of an individual peptide, or peptides, from the TUBB3 protein to the amount of another peptide, or peptides, between samples and within samples wherein the amounts as determined by signature peak area are relative one to another, regardless of the absolute weight to volume or weight to weight amounts of the TUBB3 peptide in the protein preparation from the biological sample. Relative quantitative data about individual signature peak areas between different samples are normalized to the amount of protein analyzed per sample. Relative quantitation can be performed across many peptides from multiple proteins and the TUBB3 protein simultaneously in a single sample and/or across many samples to gain insight into relative protein amounts, of one peptide/protein with respect to other peptides/proteins.

Absolute quantitative levels of the TUBB3 protein can be determined by, for example, the SRM/MRM methodology whereby the SRM/MRM signature peak area of an individual peptide from the TUBB3 protein in one biological sample is compared to the SRM/MRM signature peak area of a spiked internal standard. In one embodiment, the internal standard is a synthetic version of the same exact TUBB3 peptide that contains one or more amino acid residues labeled with one or more heavy isotopes. An isotope labeled internal standard can be synthesized so that when analyzed by mass spectrometry it generates a predictable and consistent SRM/MRM signature peak that is different and distinct from the native TUBB3 peptide signature peak and therefore can be used as a comparator peak. Thus, when the internal standard is spiked into a protein preparation from a biological sample in known amounts and analyzed by mass spectrometry, the SRM/MRM signature peak area of the native peptide is compared to the SRM/MRM signature peak area of the internal standard peptide, and this numerical comparison indicates either the absolute molarity and/or absolute weight of the native peptide present in the original protein preparation from the biological sample. Absolute quantitative data for fragment peptides are displayed according to the amount of protein analyzed per sample. Absolute quantitation can be performed across many peptides, and thus proteins, simultaneously in a single sample and/or across many samples to gain insight into absolute protein amounts in individual biological samples and in entire cohorts of individual samples.

The SRM/MRM assay method can be used to aid diagnosis of the stage of cancer, for example, directly in patient-derived tissue, such as formalin fixed tissue, and to aid in determining which therapeutic agent would be most advantageous for use in treating that patient. Cancer tissue that is removed from a patient either through surgery, such as for therapeutic removal of partial or entire tumors, or through biopsy procedures conducted to determine the presence or absence of suspected disease, is analyzed to determine whether or not a specific protein, or proteins, and which forms of proteins, are present in that patient tissue. Moreover, the expression level of a protein, or multiple proteins, can be determined and compared to a “normal” or reference level found in healthy tissue. Normal or reference levels of proteins found in healthy tissue may be derived from, for example, the relevant tissues of one or more individuals that do not have cancer. Alternatively, normal or reference levels may be obtained for individuals with cancer by analysis of relevant tissues not affected by the cancer.

Assays of protein levels (e.g., TUBB3 levels) can also be used to diagnose the stage of cancer in a patient or subject diagnosed with cancer by employing the TUBB3 levels. The level of an individual TUBB3 peptide is defined as the molar amount of the peptide determined by the SRM/MRM assay per total amount of the protein lysate analyzed. Information regarding TUBB3 can thus be used to aid in determining stage or grade of a cancer by correlating the level of the TUBB3 protein (or the fragment peptide of the TUBB3 protein) with levels observed in normal tissues. Once the quantitative amount of the TUBB3 protein has been determined in the cancer cells, that information can be matched to a list of therapeutic agents (chemical and biological) developed to specifically treat cancer tissue that is characterized by, for example, abnormal expression of the protein or protein(s) (e.g., TUBB3) that were assayed.

Improved methods of treatment of cancer also are provided by measuring TUBB3 levels in samples of formalin-fixed tumor tissue obtained from a patient and comparing the measure level against a predetermined cutoff level. When the measured TUBB3 level is below the predetermined cutoff, the patient is treated with a therapeutic regimen that includes a taxane-based therapeutic. When the measured TUBB3 level is above the cutoff, an alternative therapeutic regimen is administered as described in more detail below. Advantageously, the alternative therapeutic regimen does not contain a taxane-based therapeutic. Improved methods of treating gastric cancer and triple-negative breast cancer are provided.

Matching information from an TUBB3 protein assay to a list of therapeutic agents that specifically targets, for example, the TUBB3 protein or cells/tissue expressing the protein, defines what has been termed a personalized medicine approach to treating disease. The assay methods described herein form the foundation of a personalized medicine approach by using analysis of proteins from the patient's own tissue as a source for diagnostic and treatment decisions.

In principle, any predicted peptide derived from the TUBB3 protein, prepared for example by digesting with a protease of known specificity (e.g. trypsin), can be used as a surrogate reporter to determine the abundance of TUBB3 protein in a sample using a mass spectrometry-based SRM/MRM assay. Surprisingly, however, it has been found that many potential peptide sequences from the TUBB3 protein are unsuitable or ineffective for use in mass spectrometry-based SRM/MRM assays for reasons that are not immediately evident. This is particularly true for peptides derived from formalin fixed tissue. As it was not possible to predict the most suitable peptides for MRM/SRM assay, it was necessary to experimentally identify peptides in actual Liquid Tissue lysates to develop a reliable and accurate SRM/MRM assay for the TUBB3 protein. While not wishing to be bound by any theory, it is believed that some peptides might, for example, be difficult to detect by mass spectrometry as they do not ionize well or do not produce fragments distinct from other proteins. Peptides may also fail to resolve well in separation (e.g., liquid chromatography), or adhere to glass or plastic ware.

TUBB3 fragment peptides may be generated by a variety of means including by the use of the Liquid Tissue protocol provided in U.S. Pat. No. 7,473,532. The Liquid Tissue protocol and reagents are capable of producing peptide samples suitable for mass spectroscopic analysis from formalin fixed paraffin embedded tissue by proteolytic digestion of the proteins in the tissue/biological sample. In the Liquid Tissue protocol the tissue/biological is heated in a buffer for an extended period of time (e.g., from about 80° C. to about 100° C. for a period of time from about 10 minutes to about 4 hours) to reverse or release protein cross-linking. The buffer employed is a neutral buffer, (e.g., a Tris-based buffer, or a buffer containing a detergent). Following heat treatment the tissue/biological sample is treated with one or more proteases, including but not limited to trypsin, chymotrypsin, pepsin, and endoproteinase Lys-C for a time sufficient to disrupt the tissue and cellular structure of said biological sample and to liquefy said sample (e.g., a period of time from 30 minutes to 24 hours at a temperature from 37° C. to 65° C.). The result of the heating and proteolysis is a liquid, soluble, dilutable biomolecule lysate.

The TUBB3 peptide described below was derived from the TUBB3 protein by protease digestion of all the proteins within a complex Liquid Tissue lysate prepared from cells procured from formalin fixed cancer tissue. Unless noted otherwise, in each instance the protease was trypsin. The Liquid Tissue lysate was then analyzed by mass spectrometry to determine those peptides derived from the TUBB3 protein that are detected and analyzed by mass spectrometry. Identification of a specific preferred subset of peptides for mass-spectrometric analysis is based on; 1) experimental determination of which peptide or peptides from a protein ionize in mass spectrometry analyses of Liquid Tissue lysates, and 2) the ability of the peptide to survive the protocol and experimental conditions used in preparing a Liquid Tissue lysate.

Protein lysates from cells procured directly from formalin (formaldehyde) fixed tissue were prepared using the Liquid Tissue reagents and protocol that entails collecting cells into a sample tube via tissue microdissection followed by heating the cells in the Liquid Tissue buffer for an extended period of time. Once the formalin-induced cross linking has been negatively affected, the tissue/cells are then digested to completion in a predictable manner using a protease, such as, for example, trypsin (although other proteases can be used). Each protein lysate is turned into a collection of peptides by digestion of intact polypeptides with the protease. Each Liquid Tissue lysate was analyzed (e.g., by ion trap mass spectrometry) to perform multiple global proteomic surveys of the peptides where the data was presented as identification of as many peptides as could be identified by mass spectrometry from all cellular proteins present in each protein lysate. An ion trap mass spectrometer or another form of a mass spectrometer that is capable of performing global profiling for identification of as many peptides as possible from a single complex protein/peptide lysate is employed. Ion trap mass spectrometers however may advantageously be used conducting global profiling of peptides. Although an SRM/MRM assay can be developed and performed on any type of mass spectrometer, including a MALDI, ion trap, or triple quadrupole, advantageously a triple quadrupole instrument platform is used for an SRM/MRM assay. That type of a mass spectrometer is a suitable instrument for analyzing a single isolated target peptide within a very complex protein lysate that may consist of hundreds of thousands to millions of individual peptides from all the proteins contained within a cell.

Once as many peptides as possible were identified in a single MS analysis of a single lysate under the conditions employed, then that list of peptides was collated and used to determine the proteins that were detected in that lysate. That process was repeated for multiple Liquid Tissue lysates, and the very large list of peptides was collated into a single dataset. That type of dataset can be considered to represent the peptides that can be detected in the type of biological sample that was analyzed (after protease digestion), and specifically in a Liquid Tissue lysate of the biological sample, and thus includes one or more peptides for specific proteins, such as for example the TUBB3 protein.

In one embodiment, the TUBB3 tryptic peptide identified as useful in the determination of absolute or relative amounts of the TUBB3 protein is the peptide of SEQ ID NO:1. This peptide was detected by mass spectrometry in Liquid Tissue lysates prepared from formalin fixed, paraffin embedded tissue. This peptide may be used in a quantitative SRM/MRM assay for the TUBB3 protein in human biological samples, including directly in formalin fixed patient tissue.

SEQ ID NO: 1
ISVYYNEASSHK

This TUBB3 tryptic peptide was detected from multiple Liquid Tissue lysates of multiple different formalin fixed tissues of different human organs including lung, colon, and breast. The peptide is useful for quantitative SRM/MRM assay of the TUBB3 protein in formalin fixed tissue. Further data analysis indicates no preference is observed for any specific peptide from any specific organ site. Thus, the peptide of SEQ ID NO:1 is suitable for conducting SRM/MRM assays of the TUBB3 protein on a Liquid Tissue lysate from any formalin fixed tissue originating from any biological sample or from any organ site in the body.

In order to most efficiently implement an SRM/MRM assay for the peptide of SEQ ID NO:1 it is desirable to utilize information in addition to the peptide sequence in the analysis. That additional information may be used in directing and instructing the mass spectrometer (e.g. a triple quadrupole mass spectrometer) to perform the correct and focused analysis of specific targeted peptide(s), such that the assay may be effectively performed.

The additional information about the specific TUBB3 peptide may include one or more of the mono isotopic mass of the peptide, its precursor charge state, the precursor m/z value, the m/z transition ions, and the ion type of each transition ion. The table below shows additional peptide information that may be used to develop an SRM/MRM assay for the TUBB3 protein for using the peptide of SEQ ID NO: 1.

| | | | Pre- | | | |
|---|---|---|---|---|---|---|
| | | Mono | cursor | Pre- | Trans- | |
| SEQ | Peptide | Isotopic | Charge | cursor | ition | Ion |
| ID | sequence | Mass | State | m/z | m/z | Type |
| SEQ ID | ISVYYNEASSHK | 1396.6622 | 3 | 466.5613 | 529.2723 | y5 |
| NO: 1 | | | 3 | 466.5613 | 549.7459 | y9 + 2 |
| | | | 3 | 466.5613 | 599.2801 | y10 + 2 |
| | | | 3 | 466.5613 | 642.7961 | y11 + 2 |
| | | | 3 | 466.5613 | 772.3578 | y7 |

The method described below was used to: 1) identify candidate peptides from the TUBB3 protein that can be used for a mass spectrometry-based SRM/MRM assay for the TUBB3 protein, 2) develop an individual SRM/MRM assay, or assays, for target peptides from the TUBB3 protein in order to correlate and 3) apply quantitative assays to cancer diagnosis and/or choice of optimal therapy.

### Assay Method

1. Identification of SRM/MRM candidate fragment peptides for the TUBB3 protein
a. Prepare a Liquid Tissue protein lysate from a formalin fixed biological sample using a protease or proteases, (that may or may not include trypsin), to digest proteins
b. Analyze all protein fragments in the Liquid Tissue lysate on an ion trap tandem mass spectrometer and identify all fragment peptides from the TUBB3 protein, where individual fragment peptides do not contain any peptide modifications such as phosphorylations or glycosylations
c. Analyze all protein fragments in the Liquid Tissue lysate on an ion trap tandem mass spectrometer and identify all fragment peptides from the TUBB3 protein that carry peptide modifications such as for example phosphorylated or glycosylated residues
d. All peptides generated by a specific digestion method from the entire, full length TUBB3 protein potentially can be measured, but preferred peptides used for development of the SRM/MRM assay are those that are identified by mass spectrometry directly in a complex Liquid Tissue protein lysate prepared from a formalin fixed biological sample
e. Peptides that are specifically modified (phosphorylated, glycosylated, etc.) in patient tissue and which ionize, and thus detected, in a mass spectrometer when analyzing a Liquid Tissue lysate from a formalin fixed biological sample are identified as candidate peptides for assaying peptide modifications of the TUBB3 protein
   2. Mass Spectrometry Assay for Fragment Peptides from the TUBB3 Protein
a. SRM/MRM assay on a triple quadrupole mass spectrometer for individual fragment peptides identified in a Liquid Tissue lysate is applied to peptides from the TUBB3 protein
   i. Determine optimal retention time for a fragment peptide for optimal chromatography conditions including but not limited to gel electrophoresis, liquid chromatography, capillary electrophoresis, nano-reversed phase liquid chromatography, high performance liquid chromatography, or reverse phase high performance liquid chromatography
   ii. Determine the mono isotopic mass of the peptide, the precursor charge state for each peptide, the precursor m/z value for each peptide, the m/z transition ions for each peptide, and the ion type of each transition ion for each fragment peptide in order to develop an SRM/MRM assay for each peptide.
   iii. SRM/MRM assay can then be conducted using the information from (i) and (ii) on a triple quadrupole mass spectrometer where each peptide has a characteristic and unique SRM/MRM signature peak that precisely defines the unique SRM/MRM assay as performed on a triple quadrupole mass spectrometer
b. Perform SRM/MRM analysis so that the amount of the fragment peptide of the TUBB3 protein that is detected, as a function of the unique SRM/MRM signature peak area from an SRM/MRM mass spectrometry analysis, can indicate both the relative and absolute amount of the protein in a particular protein lysate.
   i. Relative quantitation may be achieved by:
      1. Determining increased or decreased presence of the TUBB3 protein by comparing the SRM/MRM signature peak area from a given TUBB3 peptide detected in a Liquid Tissue lysate from one formalin fixed biological sample to the same SRM/MRM signature peak area of the same TUBB3 fragment peptide in at least a second, third, fourth or more Liquid Tissue lysates from least a second, third, fourth or more formalin fixed biological samples
      2. Determining increased or decreased presence of the TUBB3 protein by comparing the SRM/MRM signature peak area from a given TUBB3 peptide detected in a Liquid Tissue lysate from one formalin fixed biological sample to SRM/MRM signature peak areas developed from fragment peptides from other proteins, in other samples derived from different and separate biological sources, where the SRM/MRM signature peak area comparison between the 2 samples for a peptide fragment are normalized to amount of protein analyzed in each sample.
      3. Determining increased or decreased presence of the TUBB3 protein by comparing the SRM/MRM signature peak area for a given TUBB3 peptide to the SRM/MRM signature peak areas from other fragment peptides derived from different proteins within the same Liquid Tissue lysate from the formalin fixed biological sample in order to normalize changing levels of TUBB3 protein to levels of other proteins that do not change their levels of expression under various cellular conditions.
      4. These assays can be applied to both unmodified fragment peptides and for modified fragment peptides of the TUBB3 protein, where the modifications include but are not limited to phosphorylation and/or glycosylation, and where the relative levels of modified peptides are determined in the same manner as determining relative amounts of unmodified peptides.
   ii. Absolute quantitation of a given peptide may be achieved by comparing the SRM/MRM signature peak area for a given fragment peptide from the TUBB3 protein in an individual biological sample to the SRM/MRM signature peak area of an internal fragment peptide standard spiked into the protein lysate from the biological sample
      1. The internal standard is a labeled synthetic version of the fragment peptide from the TUBB3 protein that is being interrogated. This standard is spiked into a sample in known amounts, and the SRM/MRM signature peak area can be determined for both the internal fragment peptide standard and the native fragment peptide in the biological sample separately, followed by comparison of both peak areas
      2. This can be applied to unmodified fragment peptides and modified fragment peptides, where the modifications include but are not limited to phosphorylation and/or glycosylation, and where the absolute levels of modified peptides can be determined in the same manner as determining absolute levels of unmodified peptides.

### 3. Apply Fragment Peptide Quantitation to Cancer Diagnosis and Treatment

a. Perform relative and/or absolute quantitation of fragment peptide levels of the TUBB3 protein and demonstrate that the previously-determined association, as well understood in the field of cancer, of TUBB3 protein expression to the stage/grade/status of cancer in patient tumor tissue is confirmed
b. Perform relative and/or absolute quantitation of fragment peptide levels of the TUBB3 protein and demonstrate correlation with clinical outcomes from different treatment strategies, wherein this correlation has already been demonstrated in the field or can be demonstrated in the future through correlation studies across cohorts of patients and tissue from those patients. Once either previously established correlations or correlations derived in the future are confirmed by this assay then the assay method can be used to determine optimal treatment strategy

Specific and unique characteristics about specific TUBB3 peptides were developed by analysis of all TUBB3 peptides on both an ion trap and triple quadrupole mass spectrometers. That information includes the monoisotopic mass of the peptide, its precursor charge state, the precursor m/z value, the transition m/z values of the precursor, and the ion types of each of the identified transitions. That information must be determined experimentally for each and every candidate SRM/MRM peptide directly in Liquid Tissue lysates from formalin fixed samples/tissue; because, interestingly, not all peptides from the TUBB3 protein can be detected in such lysates using SRM/MRM as described herein, indicating that TUBB3 peptides not detected cannot be considered candidate peptides for developing an SRM/MRM assay for use in quantitating peptides/proteins directly in Liquid Tissue lysates from formalin fixed samples/tissue.

A particular SRM/MRM assay for a specific TUBB3 peptide is performed on a triple quadrupole mass spectrometer. An experimental sample analyzed by a particular TUBB3 SRM/MRM assay is for example a Liquid Tissue protein lysate prepared from a tissue that had been formalin fixed and paraffin embedded. Data from such as assay indicates the presence of the unique SRM/MRM signature peak for this TUBB3 peptide in the formalin fixed sample.

Specific transition ion characteristics for this peptide are used to quantitatively measure a particular TUBB3 peptide in formalin fixed biological samples. These data indicate absolute amounts of this TUBB3 peptide as a function of molar amount of the peptide per microgram of protein lysate analyzed. Assessment of TUBB3 protein levels in tissues based on analysis of formalin fixed patient-derived tissue can provide diagnostic, prognostic, and therapeutically-relevant information about each particular patient. In one embodiment, this disclosure describes a method for measuring the level of the tubulin beta-3 chain protein (TUBB3) in a biological sample, comprising detecting and/or quantifying the amount of a TUBB3 fragment peptide in a protein digest prepared from the biological sample using mass spectrometry; and calculating the level of TUBB3 protein in the sample; and wherein the level is a relative level or an absolute level. In a related embodiment, quantifying the TUBB3 fragment peptide comprises determining the amount of the TUBB3 fragment peptide in a biological sample by comparison to an added internal standard peptide of known amount, wherein the internal standard peptide has the same amino acid sequence. In some embodiments the internal standard is an isotopically labeled internal standard peptide comprises one or more heavy stable isotopes selected from ¹⁸O, ¹⁷O, ³⁴S, ¹⁵N, ¹³C, ²H or combinations thereof.

The method for measuring the level of the TUBB3 protein in a biological sample described herein (or fragment peptides as surrogates thereof) may be used as a diagnostic indicator of cancer in a patient or subject. In one embodiment, the results from measurements of the level of the TUBB3 protein may be employed to determine the diagnostic stage/grade/status of a cancer by correlating (e.g., comparing) the level of TUBB3 protein found in a tissue with the level of that protein found in normal and/or cancerous or precancerous tissues

The skilled artisan will recognize that taxane agents may also be used as part of a treatment regimen that includes additional drugs or combinations of drugs. Levels of TUBB3 in patient tumor samples are typically expressed in amol/μg, although other units can be used. The skilled artisan will recognize that a reference level can be expressed as a range around a central value, for example, +/−250, 150, 100, 50 or 25 amol/μg. In the first specific example of gastric cancer described in detail below a suitable reference level for the TUBB3 protein was found to be 700 amol/μg. In the second example, of triple-negative breast cancer, the cutoff also was found to be 700 amol/μg, although cutoffs of 850 and 930 amol/μg were also found to be statistically significant. However, the skilled artisan will recognize that levels higher or lower than these reference levels can be selected based on clinical results and experience.

Improved methods of treatment are provided in which the patient is treated with a taxane-based therapeutic when the TUBB3 level is below a predetermined reference level, and where the patient is treated with an alternative regimen when the TUBB3 level is above the predetermined reference level. In the case of gastric cancer, as described below, a patient may be treated with a regimen such as FOLFIRI followed by docetaxel (Taxotere) and cisplatin (CDDP) when the TUBB3 level is below the cutoff, versus either FOLFIRI or 5-FU plus folinic acid (leucovorin) when the TUBB3 level is above the cutoff. In the case of triple negative breast cancer, the patient may be treated with ACT (anthracycline and Cytoxan, followed by taxane) when the TUBB3 level is below the cutoff, whereas the patient may be treated with CMF (Cytoxan, methotrexate and 5-FU) when the TUBB3 level is above the cutoff.

Because both nucleic acids and protein can be analyzed from the same Liquid Tissue biomolecular preparation it is possible to generate additional information about disease diagnosis and drug treatment decisions from the nucleic acids in same sample upon which proteins were analyzed. For example, if the TUBB3 protein is expressed by certain cells at increased levels, when assayed by SRM the data can provide information about the state of the cells and their potential for uncontrolled growth, choice of optimal therapy, and potential drug resistance. At the same time, information about the status of genes and/or the nucleic acids and proteins they encode (e.g., mRNA molecules and their expression levels or splice variations) can be obtained from nucleic acids present in the same Liquid Tissue biomolecular preparation. Nucleic acids can be assessed simultaneously to the SRM analysis of proteins, including the TUBB3 protein. In one embodiment, information about TUBB3 protein and/or one, two, three, four or more additional proteins may be assessed by examining the nucleic acids encoding those proteins. Those nucleic acids can be examined, for example, by one or more, two or more, or three or more of: sequencing methods, polymerase chain reaction methods, restriction fragment polymorphism analysis, identification of deletions, insertions, and/or determinations of the presence of mutations, including but not limited to, single base pair polymorphisms, transitions, transversions, or combinations thereof.

Proteomic Analysis of TUBB3 Predicts Benefit of Docetaxel Therapy in Radically Resected Adenocarcinoma of the Stomach: Reevaluation of Samples from the ITACA-S Trial

In the ITACA-S trial patients with resectable adenocarcinoma of the stomach or gastroesophageal junction were randomly assigned to either FOLFIRI (irinotecan 180 mg/m² day 1, LV 100 mg/m² as 2 h infusion and 5-FU 400 mg/m² as bolus, days 1 and 2 followed by 600 mg/m²/day as 22 h continuous infusion, q14 for four cycles) followed by docetaxel 75 mg/m² day 1, cisplatin 75 mg/m² day 1, q21 for three cycles (sequential arm) or De Gramont regimen (5-FU/LV arm). The initial conclusion from the trial was that the more intensive regimen failed to show any benefit in disease-free and OS versus monotherapy. See Ann Oncol. 25:1373-8 (2014).

The analysis described below shows use of the mass-spectrometry (MS)-based proteomics assay described above to show that low or absent expression of the Class III β-tubulin (TUBB3) protein predicts improved survival in patients treated with Taxane-based therapy.

A cutoff for TUBB3 at 700 amol/μg, as a single predictive biomarker for Taxane-based therapy was established based on the LOD of MS-based proteomics assay for TUBB3. The predictive biomarker cutoff in metastatic gastric cancer patients receiving taxane-based chemotherapy (ITACA-s trial) was clinically validated.

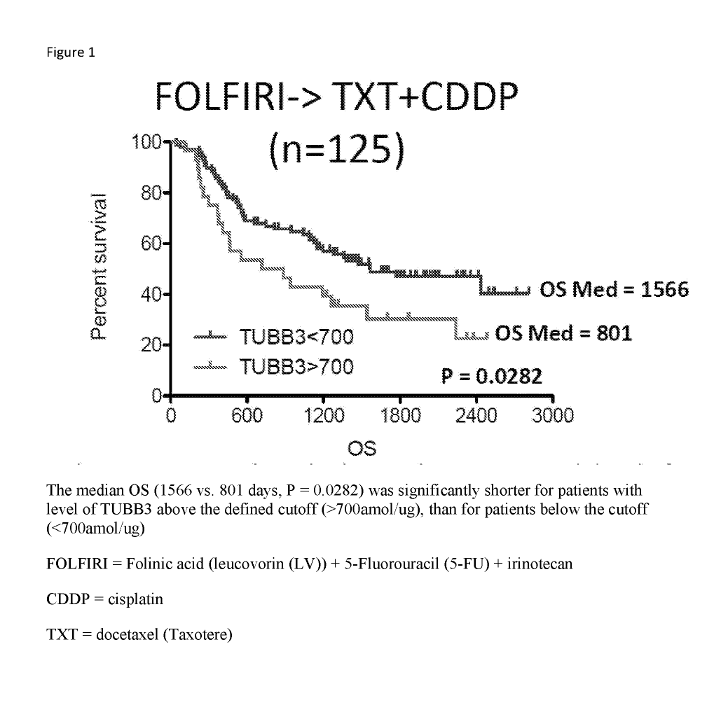

The prospectively-retrospectively declared TUBB3 cutoff in metastatic gastric cancer patients receiving taxane-based chemotherapy (ITACA-s trial) was clinically validated. The median overall survival (OS) was significantly shorter for patients with level of TUBB3 above the defined cutoff (>700 amol/μg), than for patients below the cutoff (<700 amol/μg) (1566 vs. 801 days, P=0.0282). See FIG. 1.

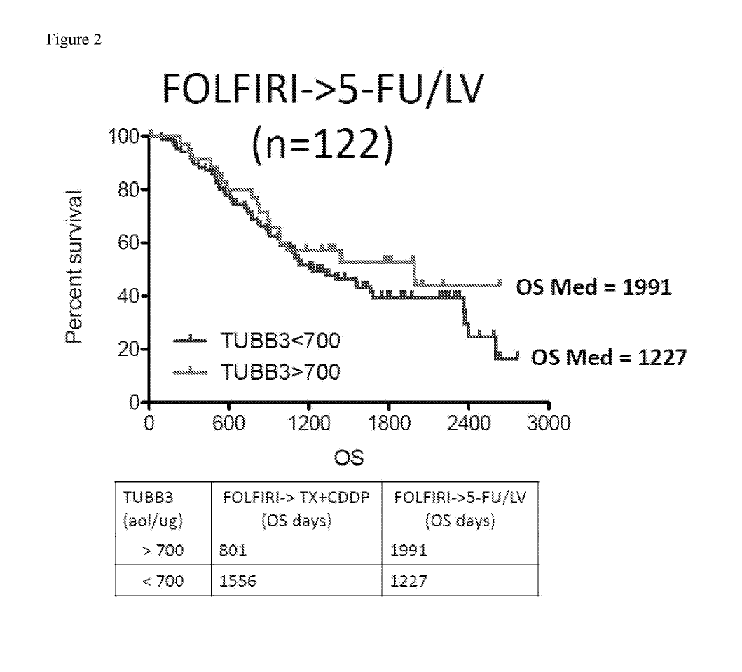

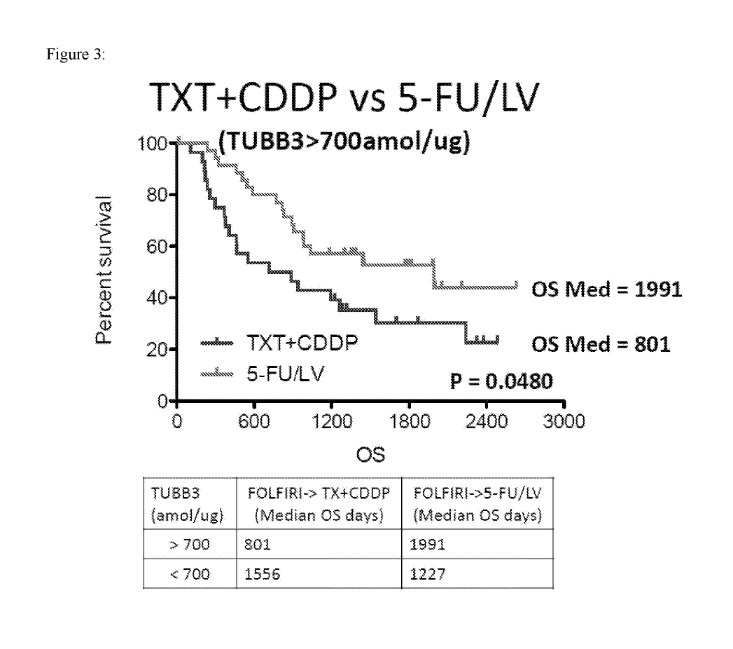

Interestingly, patients with TUBB3>700 amol/μg were found to have a significant longer OS when NOT treated with taxane-based chemotherapy compared to the patients in the taxane arm (1991 vs. 801 days, P=0.0480). Patients with TUBB3<700 amol/μg benefit from taxane-based chemotherapy compared to the patients in the 5-FU/LV arm (1556 vs. 1227 days). See FIGS. 2 and 3.

### Proteomic Analysis of TUBB3 Predicts Benefit of ACT Therapy in Triple Negative Breast Cancer:

Triple negative breast cancer (TNBC) is a heterogeneous disease with an aggressive clinical course and increased rates of local recurrence and distant metastases when compared with other breast cancer types. Joensuu et al., Ann Oncol, 23 Suppl 6: p. vi40-5 (2012). Systemic chemotherapy is often recommended to prevent a recurrence of disease but, while many chemotherapy biomarkers have been identified, screening is not routine and chemotherapy regimens are not adjusted to account for individual tumor biology. The ability to identify which patients will respond to therapy based on the biology of their individual tumors could ensure delivery of the most effective therapies while sparing patients from unnecessary toxicity.

The methods described below show the associations between tumor molecular profiles and survival among TNBC patients (n=97) treated with standard of care (ACT: doxorubicin, cyclophosphamide followed by docetaxel), using a quantitative proteomics approach.

Methods:

Microdissected tumor areas from formalin-fixed, paraffin-embedded (FFPE) tissue blocks from TNBC patients (n=97), were subjected to Liquid Tissue® digestion and proteomic analysis. A multiplexed, SRM-MS assay was used to quantitate levels of multiple proteins including TUBB3. Kaplan-Meier analysis was used to obtain optimal protein expression thresholds predictive of survival benefit after adjuvant therapy.

Results:

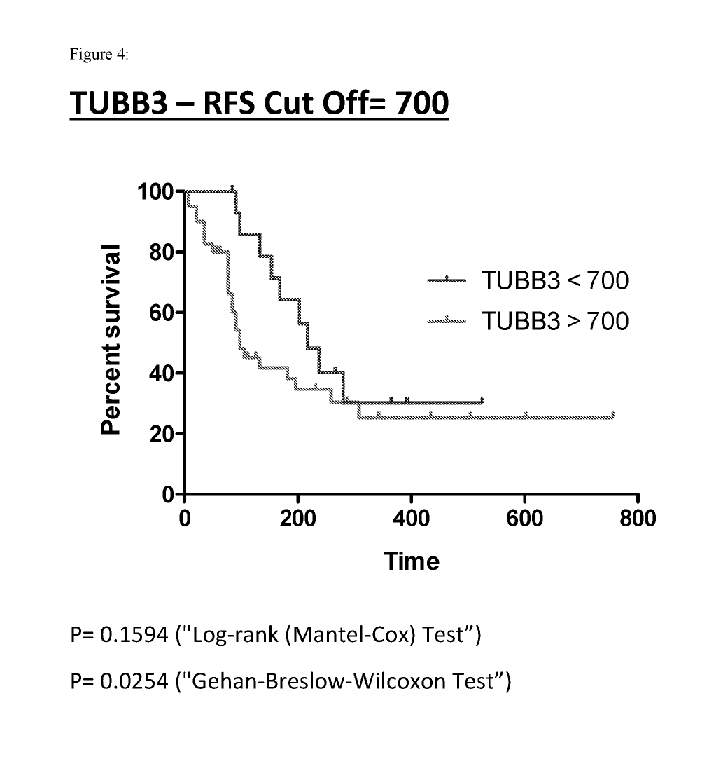

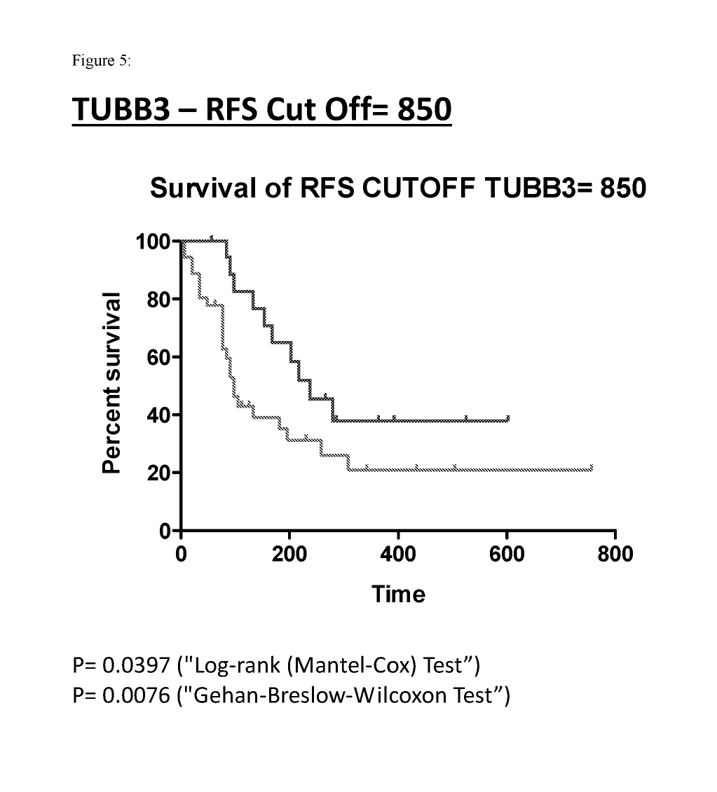

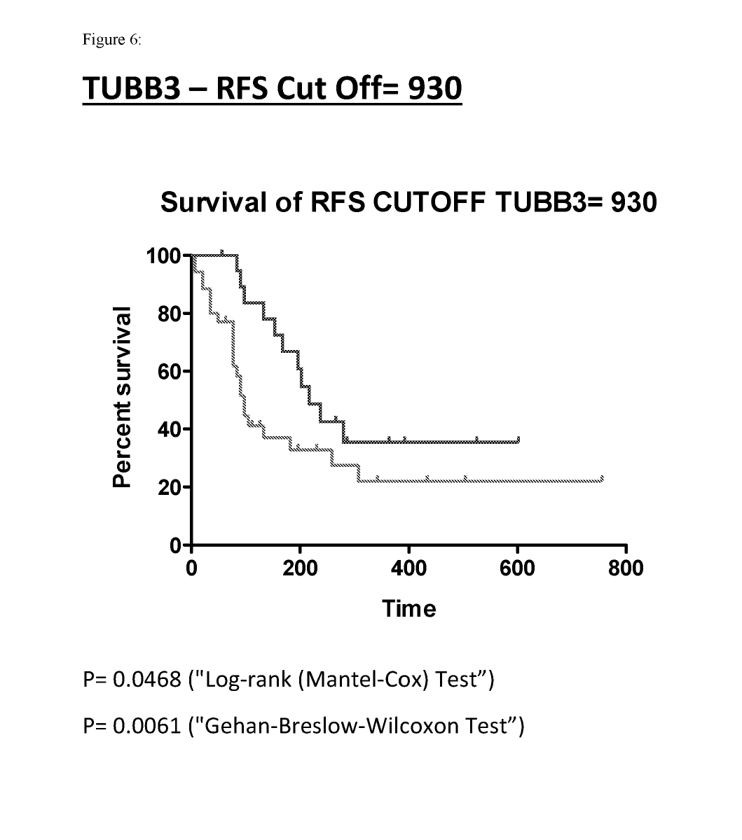

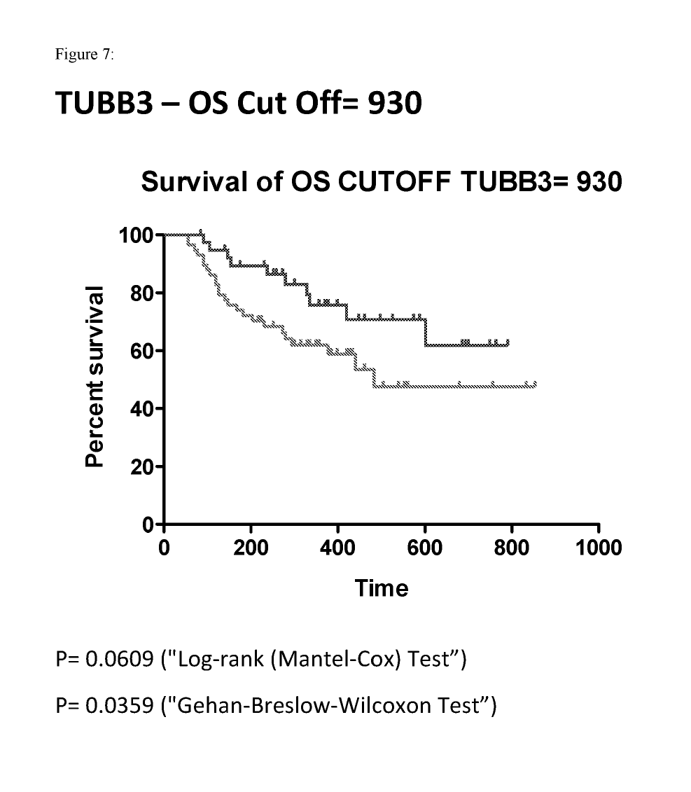

TUBB3 was detectable by targeted proteomics in 69 of the 97 patient samples (71%), and an 8.8-fold range of expression (700-6161.7 amol/μg) was measured. High protein expression of TUBB3 (TUBB3>930 amol/μg) correlated with shorter overall survival (OS) and worse relapse-free survival (RFS). RFS and overall survival (OS) were statistically significantly better in patients with low expression of TUBB3 (TUBB3<930 amol/μg). The data show that the cutoff was found to be 700 amol/μg, although cutoffs of 850 and 930 amol/μg were also found to be statistically significant. See FIGS. 4-7.

The wide expression range of TUBB3 shows that certain therapies have different response rates based on biomarker expression. The results shown here demonstrate that improved methods of treatment are provided by measuring the protein expression of TUBB3, and that TUBB3 is a predictive marker for resistance to taxane-based therapies in TNBC patients treated with ACT.

1. A method for measuring the level of the human tubulin beta-3 chain (TUBB3) protein in a human biological sample of formalin-fixed tissue, the method comprising
detecting and/or quantifying an amount of a TUBB3 fragment peptide in a protein digest prepared from said human biological sample of formalin-fixed tissue using mass spectrometry; and
calculating a level of TUBB3 protein in said sample; wherein the TUBB3 fragment peptide is SEQ ID NO:1.

2. The method of claim 1, further comprising the step of fractionating said protein digest prior to detecting and/or quantifying the amount of said TUBB3 fragment peptide.

3. (canceled)

4. The method of claim 1, wherein the tissue is paraffin-embedded tissue.

5. The method of claim 1, wherein the tissue is obtained from a tumor.

6. The method of claim 1, further comprising quantifying said TUBB3 fragment peptide.

7. The method of claim 6, wherein quantifying said TUBB3 fragment peptide comprises comparing the amount of said TUBB3 fragment peptide in one biological sample to the amount of the same TUBB3 fragment peptide in a different and separate biological sample.

8. The method of claim 7, wherein quantifying said TUBB3 fragment peptide comprises determining the amount of said TUBB3 fragment peptide in a biological sample by comparison to an added internal standard peptide of known amount, wherein said TUBB3 fragment peptide in the biological sample is compared to an internal standard peptide having the same amino acid sequence; and wherein the internal standard peptide is an isotopically labeled peptide.

9.-11. (canceled)

12. The method of claim 1, further comprising administering to the patient from which said biological sample was obtained a therapeutically effective amount of a therapeutic agent, wherein the therapeutic agent and/or amount of the therapeutic agent administered is based upon the amount of said TUBB3 fragment peptide or the level of TUBB3 protein.

13. The method of claim 1, wherein therapeutic agents bind the TUBB3 protein and/or inhibit its biological activity.

14. A method of treating a patient suffering from cancer comprising:
(a) quantifying a level of a TUBB3 fragment peptide in a protein digest prepared from a tumor sample obtained from the patient and calculating a level of the TUBB3 peptide in the tumor sample by selected reaction monitoring using mass spectrometry;
(b) comparing the level of the TUBB3 fragment peptide to a reference level, and
(c) treating the patient with a taxane-based chemotherapy regimen when the level of the TUBB3 fragment peptide is lower than the reference level; or
(d) treating the patient with a therapeutic regimen that does not comprise an effective amount of a taxane when the level of the TUBB3 fragment peptide is above the reference level.

15. The method of claim 14, wherein the reference level is 700 amol/μg., +/−250 amol/μg, of biological sample protein analyzed.

16.-22. (canceled)

23. The method of claim 14, wherein mass spectrometry comprises tandem mass spectrometry, ion trap mass spectrometry, triple quadrupole mass spectrometry, MALDI-TOF mass spectrometry, MALDI mass spectrometry, hybrid ion trap/quadrupole mass spectrometry and/or time of flight mass spectrometry.

24. The method of claim 23, wherein a mode of mass spectrometry used is Selected Reaction Monitoring (SRM), Multiple Reaction Monitoring (MRM), and/or multiple Selected Reaction Monitoring (mSRM).

25. The method of claim 14, wherein the TUBB3 fragment peptide has the amino acid sequence as set forth as SEQ ID NO:1.

26. The method of claim 14, wherein the tumor sample is a cell, collection of cells, or a solid tissue.

27. The method of claim 26, wherein the tumor sample is formalin-fixed solid tissue.

28. The method of claim 27, wherein the tissue is paraffin embedded tissue.

29. The method of claim 14, wherein quantifying the specified TUBB3 fragment peptide comprises determining the amount of the TUBB3 peptide in said sample by comparing to a spiked internal standard peptide of known amount, wherein both the native peptide in the biological sample and the internal standard peptide corresponds to the same amino acid sequence of the TUBB3 fragment peptide as shown in SEQ ID NO:1.

30. The method of claim 29, wherein the internal standard peptide is an isotopically labeled peptide.

31. The method of claim 30, wherein the isotopically labeled internal standard peptide comprises one or more heavy stable isotopes selected from ¹⁸O, ¹⁷O, ¹⁵N, ¹³C, ²H or combinations thereof.

32. The method of claim 30, wherein detecting and quantitating the specified TUBB3 fragment peptide can be combined with detecting and quantitating other peptides from other proteins in multiplex so that the treatment decision about which agent used for treatment is based upon specific levels of the specified TUBB3 fragment peptide in combination with other peptides/proteins in the biological sample.

33. The method of claim 14, wherein the patient is suffering from gastric cancer and wherein the patient is treated with FOLFIRI followed by docetaxel and cisplatin (CDDP) when the TUBB3 level is below the reference level.

34. The method of claim 14, wherein the patient is suffering from gastric cancer and wherein said patient is treated with either FOLFIRI or 5-FU plus folinic acid (leucovorin) when the TUBB3 level is above the reference level.

35. The method of claim 14, wherein the patient is suffering from triple negative breast cancer, and wherein the patient is treated with ACT (anthracycline and Cytoxan, followed by taxane) when the TUBB3 level is below the reference level or the patient is treated with CMF (Cytoxan, methotrexate and 5-FU) when the TUBB3 level is above the reference level.

36. (canceled)

37. The method of claim 35, wherein said reference level is 850 amol/μg., +/−250 amol/μg, of biological sample protein analyzed.

38.-46. (canceled)


SEQUENCE LISTING (1 sequence)

SEQ ID NO: 1 - protein, 12 aa, Homo sapiens
ISVYYNQASSHK